(19) United States
(12) Patent Application Publication
(10) Pub. No.: US 2013/0150447 A1
(43) Pub. Date: Jun. 13, 2013

(54) NOVEL OIL-IN-WATER COSMETIC EMULSIONS INCLUDING N-ACYLATED DERIVATIVES HAVING A HIGH MELTING POINT, AND METHODS FOR PREPARING SAME

(71) Applicants: Franck Barlerin, Cuq Toulza (FR); Juanshu Shen, Castres (FR)

(72) Inventors: Franck Barlerin, Cuq Toulza (FR); Juanshu Shen, Castres (FR)

(73) Assignee: SOCIETE D'EXPLOITATION DE PRODUITS POUR LES INDUSTRIES CHIMIQUES SEPPIC, Paris (FR)

(21) Appl. No.: 13/818,163

(22) PCT Filed: Sep. 20, 2011
(86) PCT No.: PCT/FR2011/052158

§ 371 Date: Feb. 21, 2013

(30) Foreign Application Priority Data
Sep. 30, 2010 (FR) 1057943

(51) Int. Cl. A61K 8/06; A61Q 19/00

(52) U.S. Cl. CPC A61K 8/062; A61Q 19/00
USPC 514/563

(57) ABSTRACT

The oil-in-water cosmetic topical emulsion contains: (1) 95 to 50 wt % of an aqueous phase (P1); and (2) 5 to 50 wt % of a fatty phase (P2), wherein the fatty phase (P2) particularly includes: 0.2 to 60 wt %, specifically 2 to 20 wt %, of at least one N-acylated amino acid derivative of formula (I): R1-C(═O)—NH—CH— (R′₁)—C(═O)—OH (I), where R1 is a saturated or unsaturated, straight or branched, aliphatic hydrocarbon radical having seven to seventeen carbon atoms, and R′₁ is a hydrogen atom or a hydrocarbon radical selected from among methyl, benzyl, isopropyl, 1-methylpropyl, or isobutyl radicals. The method for preparing the oil-in-water cosmetic topical emulsion is also described.

The present invention relates to cosmetic and/or pharmaceutical formulations comprising N-acylated derivatives of amino acids.

N-acylated derivatives of amino acids, in which the acyl radicals are derived from fatty acids comprising from eight to twenty-four carbon atoms, are commonly used, either as active ingredients or as excipients in cosmetic or pharmaceutical compositions. The European patent application published under number EP 1 449 517 discloses, for example, the use of N-lauroyl amino acids as slimming active agent.

The international applications published under numbers WO2010023390 and WO2010026325 disclose the use of N-acylated derivatives of amino acids among which are included N-palmitoyl alanine, N-palmitoyl glycine and N-palmitoyl isoleucine, respectively as an active agent for regulating the genetic profile of senescent replicating fibroblasts of human skin dermis in anti-aging compositions intended to delay the appearance of wrinkles and/or to slow down the aging of skin cells of the human body, and as an active agent for regulating the proportion of basal keratinocytes of human skin epidermis expressing the nuclear form of survivin in anti-aging compositions intended for maintaining the survival of stem cells in human skin epidermis.

Certain N-acylated derivatives have melting points above 80° C. It is therefore often necessary to solubilize them under hot conditions in an appropriate solvent, before they are used in cosmetic formulations, which excludes, for safety and environmental reasons, solvents which are effective but have a low melting point, for instance light alcohols such as ethanol, or glycols, which are known to generate volatile organic compounds when they are used at high temperature.

Other N-acylated derivatives of amino acids with a hydrophobic nature, for instance alanine, glycine, isoleucine, leucine, phenylalanine and valine, also have the characteristic of showing a lipophilic nature which requires introduction into the fatty phase of cosmetic compositions, in actual fact eliminating conventional water-soluble solvents. They must therefore be solubilized in the fatty phase of the cosmetic composition comprising them.

Furthermore, many cosmetic emulsions of oil-in-water type comprise emulsifying systems comprising surfactants which generate liquid crystals in situ, for instance alkylpolyglycosides and compositions comprising fatty alcohols and alkylpolyglycosides. However, when such oil-in-water emulsions comprise N-acylated derivatives of hydrophobic amino acids, presolubilized in the fatty phase, and a liquid-crystal-generating surfactant system, the rapid formation of lumps, therefore giving said oil-in-water emulsion a nonhomogeneous appearance, and/or the appearance, during their storage, of solid crystals comprising these N-acylated amino acids, are observed.

The inventors have therefore endeavored to develop a novel technical solution which makes it possible to prepare cosmetic compositions in the form of an oil-in-water emulsion, comprising N-acylated derivatives of amino acids of which the melting point is greater than or equal to 80° C. and comprising an emulsifying system consisting of at least one liquid-crystal-generating surfactant, and the appearance of which remains homogeneous over time, while not showing the appearance of either lumps or solid crystals.

Consequently, according to a first aspect, a subject of the invention is a composition in the form of an oil-in-water emulsion comprising, for 100% of its weight:
(1) from 95% to 50% by weight and more particularly from 90% to 70% by weight of a cosmetically acceptable aqueous phase (P1), and
(2) from 5% to 50% by weight and more particularly from 10% to 30% by weight of a fatty phase (P2),
   characterized in that said fatty phase (P2) comprises, for 100% of its weight:
a) from 0.2% to 60% by weight, more particularly from 2% to 20% by weight, of at least one N-acylated amino acid derivative of formula (I):

R₁—C(═O)—NH—CH—(R′₁)—C(═O)—OH (I)

in which R₁ represents a saturated or unsaturated, linear or branched, aliphatic hydrocarbon-based radical comprising from seven to seventeen carbon atoms, and R′₁ represents a hydrogen atom or a hydrocarbon-based radical chosen from methyl, benzyl, isopropyl, 1-methylpropyl or isobutyl radicals, and
b) from 0.2% to 80% by weight, more particularly from 2% to 40% by weight, of a solubilizing agent comprising, for 100% of its weight:
   (i) from 5% to 100% by weight of at least one compound of formula (II):

R—OH (II)
in which R represents a branched alkyl radical CH(CₙH₂ₙ₊₁)(CₘH₂ₘ₊₁)—CH₂— in which m is an even integer between 6 and 18, n is an even integer between 4 and 18, and the sum n+m is greater than 10, and
   (ii) from 0% to 95% by weight of at least one compound of formula (III):

R′O—(X)ₚ (III)

in which p represents a decimal number between 1 and 5, X represents the xylose residue and R′, which may be identical to or different than the radical R as defined above, represents a branched alkyl radical CH(CₙH₂ₙ₊₁)(CₘH₂ₘ₊₁)—CH₂— in which m is an even integer between 6 and 18, n is an even integer between 4 and 18, and the sum n+m is greater than 10;
c) from 5% to 99.4% by weight, more particularly from 5% to 94% by weight, of one or more oils and/or of one or more waxes;
d) from 0.2% to 80% by weight, more particularly from 2% to 40% by weight, of an emulsifying system (A) comprising, for 100% of its weight:
(i) from 10% to 60% by weight of at least one of the elements of the group made up of the alkylpolyglycosides of formula (IV):

R₂—O-(G)ₓ-H (IV)

in which the radical R₂ represents a saturated linear aliphatic radical comprising from twelve to twenty-two carbon atoms, G represents the residue of a reducing sugar chosen from the group consisting of glucose, xylose and arabinose, and x represents a decimal number greater than or equal to 1 and less than or equal to 5, and
(ii) from 40% to 90% by weight of at least one fatty alcohol of formula (V):

R₃—OH (V)

in which the radical R₃, which may be identical to or different than the radical R₂ as defined above, represents a saturated linear aliphatic radical comprising from 12 to 22 carbon atoms.

The N-acylated amino acid derivative of formula (I) as defined above can be in free acid form or in partial or totally salified form. When the N-acylated amino acid derivative of formula (I) is in salified form, this involves in particular alkali metal salts, such as sodium, potassium or lithium salts, alkaline-earth metal salts such as calcium, magnesium or strontium salts; an ammonium salt or amino alcohol salts such as the (2-hydroxyethyl)ammonium salt. It may also involve metal salts such as zinc or manganese divalent salts, or iron, lanthanum, cerium or aluminum trivalent salts.

In general, the level of salification of said N-acylated amino acid derivative of formula (I) will in addition depend on its pKA and on the salt concentration of the composition into which it is incorporated.

In the context of the present invention, the expression “N-acylated amino acid derivative of formula (I)” is intended to mean the N-acylated amino acid derivative of formula (I) in free form or partially or totally salified form.

The N-acylated derivative of formula (I) is generally obtained by N-acylation of an amino acid selected from the group comprising glycine, alanine, leucine, isoleucine, valine and phenylalanine or of salts thereof. The acylation reaction is known to those skilled in the art. It is described, for example, in the international application published under number WO 98/09611. It is carried out without distinction on an amino acid or on a mixture of amino acids. The acylating agent generally consists of an activated derivative of a carboxylic acid of formula:

R₁—C(═O)—OH,

in which R₁ is as defined previously, such as a symmetrical anhydride of this acid, the methyl ester of this acid, or an acid halide, for instance the acid chloride or the acid bromide.

It may also consist of a mixture of activated derivatives of carboxylic acids derived from natural oils or fats of animal or plant origin, such as copra oil, palm kernel oil, palm oil, soybean oil, rapeseed oil, corn oil, beef tallow, spermaceti oil or herring oil.

In the context of the present invention, the mixtures of fatty acids derived from copra oil, from palm kernel oil, from spermaceti oil or from coconut oil which contain a major fraction of dodecanoic acid are preferably used:

| | Copra oil | Palm kernel oil | Spermaceti oil | Coconut oil |
|---|---|---|---|---|
| Octanoic or caprylic | 6% to 9% | 3% to 10% | — | 2% to 10% |
| acid (C₈H₁₆O₂) | | | | |
| Decanoic or capric | 6% to 10% | 3% to 14% | 1% to 3% | 3% to 9% |
| acid (C₁₀H₂₀O₂) | | | | |
| Dodecanoic or lauric | 44% to 51% | 37% to 52% | 14% to 38% | 45% to 56% |
| acid (C₁₂H₂₄O₂) | | | | |
| Tetradecanoic or | 13% to 18% | 7% to 17% | 12% to 14% | 15% to 23% |
| myristic acid | | | | |
| (C₁₄H₂₈O₂) | | | | |
| Hexadecanoic or | 8% to 10% | 2% to 9% | 8% to 10% | 7% to 14% |
| palmitic acid | | | | |
| (C₁₆H₃₂O₂) | | | | |
| Octadecanoic or | 1% to 3% | 1% to 3% | 1% to 3% | 1% to 7% |
| stearic acid | | | | |
| (C₁₈H₃₆O₂) | | | | |
| Octadecanoic or | 5.5% to 7.5% | 11% to 23% | 15% to 18% | 3% to 11% |
| oleic acid | | | | |
| (C₁₈H₃₄O₂) | | | | |
| Eicosenoic or | — | — | 5% to 8% | — |
| gadolic acid | | | | |
| (C₂₀H₃₈O₂) | | | | |
| Octadecadienoic | <2.5% | 1% to 3% | — | ≦3% |
| or linoleic | | | | |
| acid (C₁₈H₃₂O₂) | | | | |
| Other acids | <0.4% | <0.6% | 26% to 34% | <3% |

According to one particular aspect, a subject of the invention is a composition as defined previously, characterized in that, in formula (I), the radical R₁—C(═O)— represents a radical chosen from octanoyl, decanoyl, dodecanoyl, tetradecanoyl, hexadecanoyl, octadecanoyl or octadecenoyl radicals.

According to another particular aspect, a subject of the invention is a composition as defined previously, characterized in that, in formula (I), the radical R′₁, represents a hydrogen atom or a hydrocarbon-based radical chosen from methyl, isopropyl, 1-methylpropyl or isobutyl radicals, and more particularly a hydrogen atom or a hydrocarbon-based radical chosen from methyl, 1-methylpropyl or isobutyl radicals.

According to the latter particular aspect, the compound of formula (I) is chosen from N-palmitoyl alanine, N-palmitoyl glycine, N-palmitoyl leucine, N-palmitoyl isoleucine or N-cocoyl alanine.

The term “cocoyl radical” is intended to mean, in the disclosure of the present invention, a radical derived from the mixture of fatty acids originating from coconut oil, the composition of which is described in the previous table.

According to another particular aspect, a subject of the invention is the composition as defined previously, for which, in formulae (II) and (III), the radicals R and R′, which may be identical or different, represent a radical chosen from 2-hexyldecyl, 2-hexyldodecyl, 2-octyldecyl, 2-octyldodecyl and 2-decyltetradecyl radicals.

The compounds of formula (II), which are constituents of the solubilizing agent in the fatty phase (P2) of the cosmetic emulsion which is a subject of the present invention, are branched alcohols obtained according to the Guerbet process consisting of a chemical reaction for converting a linear aliphatic alcohol into a β-alkyl dimeric alcohol with the loss of one mol of water. These compounds of formula (II) are sold under the brand name ISOFOL™ by the company SASOL and under the brand name EUTANOL™ by the company COGNIS. In the definition of the compounds of formula (III) as defined previously, p is a decimal number which represents the average degree of polymerization of the xylose residue. When p is an integer, (X)p is the polymer residue of rank p of the xylose residue. When p is a decimal number, the formula (III) represents a mixture of compounds:

a₁R′—O—(X)₁—H+a₂R′—O—(X)₂—H+a₃R′—O—(X)₃—H+ . . . +a_{q}R′—O—(X)_{q}—H

with q representing an integer between 1 and 5 and in the molar proportions a₁, a₂, a₃, . . . a_{q} such that:

$${\sum }_{q = 5}^{q = 1} {a}_{q} = 1 ; {a}_{1} > 0$$

According to another particular aspect of the present invention, in the definition of the compounds of formulae (I), p is between 1.05 and 5, and more particularly between 1.05 and 2.

The oligomeric structure (X)ₚ can be in any form of isomerism, whether it is optical isomerism, geometric isomerism or positional isomerism; it can also represent mixtures of isomers. In formula (III) as defined above, the radical R′—O— is bonded to X via the anomeric carbon of the saccharide residue, so as to form an acetal function.

The compounds of formula (III) are prepared by reacting xylose with an excess of alcohol of formula R′—OH, for which R′ represents a branched alkyl radical CH(CₙH₂ₙ₊₁)(CₘH₂ₘ₊₁)—CH₂— in which m is an even integer between 6 and 18, n is an even integer between 4 and 18, and the sum n+m is greater than 10. This reaction is generally carried out in a reactor in the presence of an acidic catalytic system, while controlling the stoichiometric ratio between the two reactants, and with mechanical stirring under predetermined temperature and partial vacuum conditions, for example at a temperature of between 70° C. and 130° C. and under a partial vacuum of between 300 mbar (3×10⁴ Pa) and 20 mbar (2×10³ Pa). The term “acidic catalytic system” denotes strong acids, such as sulfuric acid, hydrochloric acid, phosphoric acid, nitric acid, hypophosphorous acid, methanesulfonic acid, para-toluenesulfonic acid, or (trifluoromethane)sulfonic acid, or ion exchange resins. The unreacted alcohol of formula R′—OH is eliminated according to methods known to those skilled in the art, for instance distillation, thin-film distillation, molecular distillation or solvent extraction. Such a preparation process can be completed, if necessary or if desired, with neutralization, filtration or discoloration operations.

In the composition in the form of an oil-in-water cosmetic emulsion which is the subject of the present invention and as defined previously, among the constituent oils of the fatty phase (P2), mention may be made of:
mineral oils such as liquid paraffin, liquid petroleum jelly, isoparaffins and white mineral oils;
oils of animal origin, such as squalene or squalane;
plant oils, such as phytosqualane, sweet almond oil, copra oil, castor oil, jojoba oil, olive oil, rapeseed oil, groundnut oil, sunflower oil, wheatgerm oil, corn germ oil, soybean oil, cottonseed oil, alfalfa oil, poppy oil, pumpkin oil, evening primrose oil, millet oil, barley oil, rye oil, safflower oil, candlenut oil, passionflower oil, hazelnut oil, palm oil, shea butter, apricot kernel oil, beauty leaf oil, sysymbrium oil, avocado oil, calendula oil, sesame oil, meadowsweet oil, macadamia/kiwi oil, borage oil, blackcurrant oil, coffee oil, pistachio oil, peach kernel oil, raspberry seed oil, strawberry seed oil, melon oil, blueberry seed oil, argan oil, oily prune extract, pomegranate oil, papaya oil, coconut milk oil, oils derived from flowers or from vegetables;
ethoxylated plant oils;
synthetic oils, for instance fatty acid esters, such as butyl myristate, propyl myristate, cetyl myristate, isopropyl palmitate, butyl stearate, hexadecyl stearate, isopropyl stearate, octyl stearate, isocetyl stearate, dodecyl oleate, hexyl laurate, dicaprylyl ether, propylene glycol dicaprylate, lanolic acid-derived esters, such as isopropyl lanolate, isocetyl lanolate, fatty acid monoglycerides, diglycerides and triglycerides, for instance glyceryl triheptanoate, alkyl benzoates, hydrogenated oils, poly-alpha-olefins, polyolefins such as polyisobutene, synthesis isoalkanes, hydrogenated polydecene or hydrogenated polyisobutene, sold in France by the company Ets B. Rossow et Cie under the name PARLEAM-POLYSYNLANE™, mentioned in: Michel and Irene Ash; Thesaurus of Chemical Products, Chemical Publishing Co, Inc. 1986 Volume I, page 211 (ISBN 0 7131 3603 0), perfluoro oils; and
silicone oils, for instance dimethylpolysiloxanes, methylphenylpolysiloxanes, silicones modified with amines, silicones modified with fatty acids, silicones modified with alcohols, silicones modified with alcohols and fatty acids, silicones modified with polyether groups, modified epoxy silicones, silicones modified with fluoro groups, cyclic silicones and silicones modified with alkyl groups.

In the composition in the form of an oil-in-water cosmetic emulsion which is the subject of the present invention and as defined previously, among the constituent waxes of the fatty phase (P2), mention may be made of beeswax, carnauba wax, candelilla wax, ouricoury wax, Japan wax, cork fiber wax, sugar cane wax, jojoba wax, blackcurrant flower wax, narcissus flower wax, orange flower wax, orange wax, rice wax, paraffin waxes, lignite waxes, microcrystalline waxes, lanolin wax; ozokerite; polyethylene wax, silicone waxes; plant waxes; fatty alcohols and fatty acids that are solid at ambient temperature; glycerides that are solid at ambient temperature.

In the definition of the compounds of formula (IV) which are present in the emulsifying system (A) included in the composition in the form of an oil-in-water cosmetic emulsion which is the subject of the invention and as defined previously, x is a decimal number which represents the average degree of polymerization of the residue G.

When x is an integer, (G)ₓ is the polymer residue of rank x of the residue G. When x is a decimal number, formula (IV) represents a mixture of compounds:

a₁R₂—O-(G)₁-H+a₂R₂—O-(G)₂-H+a₃R₂—O-(G)₃-H+ . . . +a_{q}R₂—O-(G)_{q}-H

with q representing an integer between 1 and 5 and in the molar proportions a₁, a₂, a₃, . . . a_{q} such that:

$${\sum }_{q = 5}^{q = 1} {a}_{q} = 1 ; {a}_{1} > 0$$

According to another particular aspect of the present invention, in the definition of the compounds of formulae (IV), x is between 1.05 and 5, and more particularly between 1.05 and 2.

The term “residue of a reducing sugar” denotes, for G in the definition of the compounds of formulae (IV), a residue of saccharide derivatives which do not have in their structures a glycosidic linkage established between an anomeric carbon and the oxygen of an acetal group, as they are defined in the reference work: “Biochemistry”, Daniel Voet/Judith G. Voet, p. 250, John Wyley & Sons, 1990. The oligomeric structure (G)ₓ can be in any form of isomerism, whether it is optical isomerism, geometric isomerism or positional isomerism; it can also represent mixtures of isomers.

In formula (IV) as defined above, the radical R₂ is linked to G via the anomeric carbon of the saccharide residue, so as to form an acetal function.

According to one particular aspect of the present invention, the term “residue of a reducing sugar” denotes, in the definition of the compound of formula (IV) as defined above, the glucose residue.

According to a more particular aspect of the present invention, in the definition of the compound of formula (IV) included in the emulsifying system (A) contained in the composition in the form of an oil-in-water cosmetic emulsion which is the subject of the present invention, the radical R₂ is chosen from dodecyl, tetradecyl, hexadecyl, octadecyl and 12-hydroxystearyl radicals.

The compounds of formula (IV) are prepared by reacting the reducing sugar G-OH with an excess of alcohol of formula R₂—OH, for which R₂ represents a saturated linear aliphatic radical comprising from 12 to 22 carbon atoms. This reaction is generally carried out in a reactor in the presence of an acidic catalytic system, while controlling the stoichiometric ratio between the two reactants, and with mechanical stirring under predetermined temperature and partial vacuum conditions, for example at a temperature of between 70° C. and 130° C. and under a partial vacuum of between 300 mbar (3×10⁴ Pa) and 20 mbar (2×10³ Pa). The term “acidic catalytic system” denotes strong acids such as sulfuric acid, hydrochloric acid, phosphoric acid, nitric acid, hypophosphorous acid, methanesulfonic acid, para-toluenesulfonic acid or (trifluoromethane)sulfonic acid, or ion exchange resins. The unreacted alcohol of formula R₂—OH is eliminated according to methods known to those skilled in the art, for instance distillation, thin-film distillation, molecular distillation or solvent extraction. Such a preparation process can be completed, if necessary or if desired, with neutralization, filtration and discoloration operations.

According to a more particular aspect of the present invention, in the definition of the fatty alcohols of formula (V) included in the emulsifying system (A) contained in the composition in the form of an oil-in-water cosmetic emulsion which is the subject of the present invention, the radical R₃ is chosen from dodecyl, tetradecyl, hexadecyl, octadecyl and 12-hydroxystearyl radicals.

When the emulsifying system (A) comprises both a compound of formula (IV) as defined above or a mixture of compounds of formula (IV) and a compound of formula (V) as defined above or a mixture of compounds of formula (V), with the radicals R₂ and R₃ being identical, a first pathway for preparing the emulsifying system (A) consists in mixing, in the desired weight proportions, the compound of formula (IV) or a mixture of compounds of formula (IV), with the compound of formula (V) or a mixture of compounds of formula (V).

A second pathway for preparing an emulsifying system as described previously consists in carrying out a process of reacting the reducing sugar G-OH with an excess of alcohol of formula R₂—OH, generally carried out in a reactor in the presence of an acidic catalytic system, as described previously for the preparation of the compound of formula (IV), while controlling the stoichiometric ratio between the two reactants, and under predetermined temperature and partial vacuum conditions, as described previously for the preparation of the compound of formula (IV). The control of the stoichiometric ratio between the two reactants makes it possible to dispense with the step of eliminating the alcohol of formula R₂—OH as described previously for the preparation of the compound of formula (IV), and such a preparation process can be completed, if necessary or if desired, with neutralization, filtration and discoloration operations.

When the emulsifying system (A) comprises both a compound of formula (IV) as defined above or a mixture of compounds of formula (IV) and a compound of formula (V) as defined above or a mixture of compounds of formula (V), with the radicals R₂ and R₃ being different, a pathway for preparing the emulsifying system (A) consists in mixing, in the desired weight proportions, the compound of formula (IV) or a mixture of compounds of formula (IV), with the compound of formula (V) or a mixture of compounds of formula (V).

The expression “cosmetically acceptable” used in the definition of the composition which is the subject of the present invention means, according to European Economic Community Council Directive No. 76/768/EEC of Jul. 27, 1976, amended by Directive No. 93/35/EEC of Jun. 14, 1993, any substance or preparation intended for placing in contact with the various parts of the human body (epidermis, hair system, nails, lips and genital organs) or with the teeth and the mucus membranes of the oral cavity with a view exclusively or principally to cleaning them, perfuming them, changing their appearance and/or correcting body odors and/or protecting them or keeping them in good condition. A cosmetically acceptable medium of these compositions which are the subject of the invention can conventionally contain water, one or more cosmetically acceptable organic solvents, or a mixture of water and one or more organic solvents. The cosmetically acceptable solvents can more particularly be chosen from polyhydric alcohols, for instance glycerol, diglycerol, glycerol oligomers, ethylene glycol, propylene glycol, hexylene glycol, diethylene glycol, xylitol, erythritol or sorbitol, or water-soluble alcohols.

According to another particular aspect of the present invention, the composition as defined previously also comprises, for 100% of its weight, from 0.1% to 5% by weight, more particularly from 0.1% to 2% by weight, of a thickening and/or gelling polymer.

Among the thickening and/or gelling polymers that can be combined with the composition in the form of an oil-in-water cosmetic emulsion as defined previously and which is the subject of the present invention, mention may be made of:
polymers of polyelectrolyte type, for instance copolymers of acrylic acid and of 2-methyl-[(1-oxo-2-propenyl)amino]-1-propanesulfonic acid (AMPS), copolymers of acrylamide and of 2-methyl-[(1-oxo-2-propenyl)amino]-1-propanesulfonic acid, copolymers of 2-methyl-[(1-oxo-2-propenyl)amino]-1-propanesulfonic acid and of 2-hydroxyethyl acrylate, the homopolymer of 2-methyl-[(1-oxo-2-propenyl)amino]-1-propanesulfonic acid, the homopolymer of acrylic acid, copolymers of acryloylethyltrimethylammonium chloride and of acrylamide, copolymers of AMPS and of vinylpyrrolidone, copolymers of acrylic acid and of alkyl acrylates of which the carbon chain comprises between ten and thirty carbon atoms, copolymers of AMPS and of alkyl acrylates of which the carbon chain comprises between ten and thirty carbon atoms. Such polymers are sold respectively under the names SIMULGEL™ EG, SEPIGEL™ 305, SIMULGEL™ NS, SIMULGEL™ 800, SIMULGEL™ A, SIMULGEL™ EPG, SIMULGEL™ INS, SIMULGEL™ FL, SIMULGEL™ SMS 88, SEPIGEL™ 501, SEPIGEL™ 502, SEPIPLUS™ 250, SEPIPLUS™ 265, SEPIPLUS™ 400, SEPIPLUS™ S, SEPINOV™ EMT 10, SEPIMAX™ ZEN, CARBOPOL™, ULTREZ™ 10, ACULYN™, PEMULEN™ TR1, PEMULEN™ TR2, LUVIGEL™ EM, SALCARE™ SC91, SALCARE™ SC92, SALCARE™ SC95, SALCARE™ SC96, FAOCARE™ ET100, FAOCARE™ ET58, HISPAGEL™, NOVEMER™ EC1, ARISTOFLEX™ AVC, ARISTOFLEX™ HBM, RAPITHIX™ A60, RAPITHIX™ A100, COSMEDIA SP and STABILEZE™ 06;
hydrocolloids of plant or biosynthetic origin, for example xanthan gum, karaya gum, carraghenates, alginates and galactomannans;
silicates; cellulose and derivatives thereof; starch and hydrophilic derivatives thereof; polyurethanes.

The compositions in the form of an oil-in-water emulsion as defined previously and which are subjects of the present invention can be in the form of creams, milks, cream gels, fluid lotions, or vaporizable fluid lotions.

In general, these compositions in the form of oil-in-water emulsions as defined previously and which are subjects of the present invention are administered topically to human beings or to animals. The expression “topically” used means that said composition is employed by application to the skin, the hair, the scalp or the mucus membranes, whether it is a direct application in the case of a dermocosmetic, cosmetic composition, or an indirect application for example in the case of a body hygiene product in the form of a textile or paper wipe or of sanitary products intended to be in contact with the skin or the mucus membranes.

In general, these compositions in the form of oil-in-water emulsions as defined previously and which are subjects of the present invention also comprise excipients and/or active ingredients normally used in the field of formulations for topical use, in particular cosmetic, dermocosmetic, pharmaceutical or dermopharmaceutical formulations, such as thickening and/or gelling surfactants, stabilizers, film-forming compounds, hydrotropic agents, plasticizers, emulsifiers and coemulsifiers, opacifiers, nacreous agents, overfatting agents, sequestrants, chelating agents, antioxidants, fragrances, preservatives, conditioning agents, bleaching agents intended for decolorizing bodily hair and the skin, active ingredients intended to provide a treating action with respect to the skin or the hair, sunscreens, inorganic fillers or pigments, particles that afford a visual effect or that are intended for encapsulating active agents, exfoliant particles, texturing agents, optical brighteners and insect repellants.

As examples of thickening and/or gelling surfactants optionally present in the compositions in the form of oil-in-water emulsions which are subjects of the present invention, mention may be made of:
optionally alkoxylated fatty esters of alkylpolyglycosides, and quite particularly ethoxylated methylpolyglucoside esters such as PEG 120 methyl glucose trioleate and PEG 120 methyl glucose dioleate sold, respectively, under the names GLUCAMATE™ LT and GLUMATE™ DOE120;
alkoxylated fatty esters such as PEG 150 pentaerythrityl tetrastearate sold under the name CROTHIX™ DS53, PEG 55 propylene glycol oleate sold under the name ANIIL™ 141;
fatty-chain polyalkylene glycol carbamates such as PPG 14 laureth isophoryl dicarbamate sold under the name ELFACOS™ T211, PPG 14 palmeth 60 hexyl dicarbamate sold under the name ELFACOS™ GT2125.

As examples of emulsifiers optionally present in the compositions in the form of oil-in-water emulsions which are subjects of the present invention, mention may be made of:—optionally alkoxylated alkylpolyglycoside fatty esters, and quite particularly ethoxylated methylpolyglucoside esters such as PEG 120 methyl glucose trioleate and PEG 120 methyl glucose dioleate sold, respectively, under the names GLUCAMATE™ LT and GLUMATE™ DOE120;
alkoxylated fatty esters such as PEG 150 pentaerythrityl tetrastearate sold under the name CROTHIX™ DS53, PEG 55 propylene glycol oleate sold under the name ANIIL™ 141;
fatty-chain polyalkylene glycol carbamates such as PPG 14 laureth isophoryl dicarbamate sold under the name ELFACOS™ T211, PPG 14 palmeth 60 hexyl dicarbamate sold under the name ELFACOS™ GT2125;
fatty acids, ethoxylated fatty acids, fatty acid esters of sorbitol, fatty acid esters of mannitol, ethoxylated fatty acid esters, polysorbates, glyceryl esters such as glyceryl stearate and the composition comprising glyceryl stearate and PEG-100 stearate sold by the company SEPPIC under the name SIMULSOL™ 165, polyglyceryl esters such as polyglyceryl polyhydroxystearates, polyglycol esters such as polyglycol polyhydroxystearates, polyol esters, ethoxylated fatty alcohols, sucrose esters, fatty alcohol sulfates and fatty alcohol phosphates.

As examples of opacifiers and/or nacreous agents optionally present in the compositions in the form of oil-in-water emulsions which are subjects of the present invention, mention may be made of sodium or magnesium palmitates or stearates or hydroxystearates, ethylene or polyethylene glycol monostearates or distearates, fatty alcohols, styrene homopolymers and copolymers such as the styrene acrylate copolymer sold under the name MONTOPOL™ OP1 by the company SEPPIC.

As examples of texturing agents optionally present in the compositions in the form of oil-in-water emulsions which are subjects of the present invention, mention may be made of N-acylated derivatives of amino acids, such as the lauroyl lysine sold under the name AMINOHOPE™ LL by the company AJINOMOTO, the poly(methylmethacrylate)s sold under the name MICROPEARL™ by the company SEPPIC, and nylon-12.

As examples of sunscreens optionally present in the compositions in the form of oil-in-water emulsions which are subjects of the present invention, mention may be made of all those which appear in amended cosmetic directive 76/768/EEC annex VII.

As examples of active ingredients optionally present in the compositions in the form of oil-in-water emulsions which are subjects of the present invention, mention may be made of: compounds with a lightening or depigmenting action, for instance arbutin, kojic acid, hydroquinone, ellagic acid, vitamin C or vitamin C derivatives, for instance magnesium ascorbyl phosphate, polyphenol extracts, grape extracts, pine extracts, wine extracts, olive extracts, marc extracts, apple juice extracts, amino acid derivatives, for instance the undecelenoyl phenylalanine sold under the name SEPIWHITE MSH, peptides; total protein hydrolyzates; partial protein hydrolyzates; polyols (for instance glycerol or butylene glycol); urea; hydroxyethylurea; pyrrolidonecarboxylic acid or derivatives of this acid; glycyrrhetinic acid; α-bisabolol; sugars or sugar derivatives; polysaccharides or derivatives thereof; hydroxy acids, for example lactic acid; vitamins or vitamin derivatives, for instance retinol, vitamin E and derivatives thereof; mineral salts; enzymes; coenzymes, for instance coenzyme Q10; hormones or “hormone-like” compounds, for instance PHYTO-AGE™; soybean extracts, for instance Raffermine™, wheat extracts, for instance Tensine™ or Gliadine™; plant extracts such as tannin-rich extracts, isoflavone-rich extracts or terpene-rich extracts; extracts of fresh water algae or of marine algae; extracts of marine plants; essential waxes; bacterial extracts; lipids in general and more particularly lipids such as ceramides or phospholipids; active agents with antimicrobial activity or a purifying action on greasy skin; active agents with an energizing or stimulating property, for instance SEPITONIC™ M3 or Physiogenyl™; panthenol and derivatives thereof, for instance SEPICAP™ MP; anti-aging active agents, for instance SEPIVINOL™, SEPIVITAL™, EXTRAMEL™ C, and MANOLIVA™; moisturizing active agents such as AQUAXYL™; “anti-photoaging” active agents; active agents with an immediate tensioning or smoothing action on the skin, for instance SESAFLASH™; active agents for protecting the integrity of the dermal-epidermal junction, for instance PHYTO-AGE™; active agents for increasing the synthesis of extracellular matrix components, for instance SEPITONIC™ M3 and AQUAXYL™; active agents with slimming, firming or draining activity, for instance caffeine, caffeine derivatives, theophylline, cyclic adenosine monophosphate (cAMP), ADIPOLESS™, green tea, sage, gingko biloba, ivy, common horse chestnut, bamboo, ruscus, butcher's broom, Centella asiatica, heather, meadowsweet, fucus, rosemary, willow, parsnip extracts, potentilla extracts; active agents that create a “heating” sensation on the skin, such as cutaneous capillary circulation activators, for instance nicotinates; active agents that create a “freshness” sensation on the skin, for instance menthol and derivatives thereof; active agents with an action on stem cells; active agents with an action on the epidermis, the dermis, the hypodermis and the cutaneous appendages (bodily hair, nails, sebaceous glands, pores, etc.); active agents with an action on the cutaneous flora; agents for artificial tanning and/or browning of the skin, for instance monocarbonyl or polycarbonyl compounds, such as dihydroxyacetone, isatin, alloxan, ninhydrin, glyceraldehyde, mesotartaric aldehyde, glutaraldehyde or erythrulose.

A subject of the invention is also a process for preparing the composition as defined previously, characterized in that it comprises:
at least one step a) of preparing said fatty phase (P2) by mixing, for 100% by weight of said phase (P2):
1) from 0.2% to 60% by weight, more particularly from 2% to 20% by weight, of at least one N-acylated amino acid derivative of formula (I), with
2) from 0.2% to 80% by weight, more particularly from 2% to 40% by weight, of said solubilizing agent;
3) from 5% to 99.4% of one or more oils and/or of one or more waxes and with
4) from 0.2% to 80% by weight, more particularly from 2% to 40% by weight, of said emulsifying system (A); and
5) optionally a thickening and/or gelling polymer, in the amount required to obtain, in said composition, from 0.1% to 5% by weight, more particularly from 0.1% to 2% by weight, of said thickening and/or gelling polymer,
at least one step b) of emulsifying from 5% to 50% by weight of said fatty phase P2 in from 95% to 50% by weight of said aqueous phase P1.

According to one particular aspect, step a) of preparing the fatty phase (P2) is carried out at a temperature of between 70° C. and 90° C., more particularly between 80° C. and 90° C.

In the process which is the subject of the invention, step a) of preparing the fatty phase (P2) as described above can be carried out by means of any mixing device known to those skilled in the art, for instance by means of a stirring device equipped with a spindle of “anchor” type, at stirring speeds of between 80 revolutions/minute and 1000 revolutions/minute, and for instance by means of a stirring device of rotor-stator type at stirring speeds of between 1000 revolutions/minute and 8000 revolutions/minute.

In the process as defined previously, emulsifying step b) is advantageously carried out at a temperature of between 70° C. and 90° C., more particularly between 80° C. and 90° C., and by means of a stirring device of rotor-stator type at stirring speeds of between 1000 revolutions/minute and 8000 revolutions/minute.

A subject of the invention is also the use of the composition as defined previously, as a cosmetic topical composition via its application to the skin or the mucus membranes of the human body.

The following examples illustrate the invention without, however, limiting it.

### EXAMPLE 1

### Preparation of Oil-in-Water Emulsions Comprising an N-Acylated Derivative of Formula (I) According to the Invention and According to the Prior Art

A series of oil-in-water emulsions (emulsions E1 to E3), the compositions of which are indicated in table 1 below, are prepared by carrying out the following in the following process:

Step a):

The emulsifying system is added to the oils previously introduced into a reactor brought to a temperature of 85° C. and the mixture obtained is homogenized for 30 minutes by means of a stirrer equipped with a spindle of anchor type, at a speed of 80 revolutions/minute. The N-acylated derivative of formula (I) is then added to this mixture with the solubilizing agent at a temperature of 85° C., and the resulting fatty phase is homogenized for 30 minutes by means of a stirrer equipped with a spindle of anchor type, at a speed of 80 revolutions/minute.

Step b):

The thickening polymer is introduced into the mixture prepared at the end of step a) maintained at a temperature of 85° C. The resulting mixture is homogenized for 15 minutes by means of a stirrer equipped with a spindle of anchor type, at a speed of 80 revolutions/minute.

Step c):

The ingredients of the aqueous phase are added to the mixture prepared at the end of step b), and the resulting mixture is maintained at a temperature of 85° C., then subjected to stirring by means of an emulsifying device of rotor-stator type, sold by the company SILVERSON, for a period of 4 minutes at a speed of 4000 revolutions/minute. The mixture resulting from step c) is then cooled to ambient temperature.

TABLE 1
Composition of emulsions E1 to E3.

| | Emulsion E1 | | |
|---|---|---|---|
| | (according | Comparative | Comparative |
| | to the | emulsion | emulsion |
| | invention) | E2 | E3 |
| Fatty phase | | | |
| Emulsifying system: | | | |
| Montanov ™ L⁽¹⁾ | 4% | 4% | 4% |
| Oils: | | | |
| Cera Alba | 3% | 3% | 3% |
| Butyrospermum Parkii Shea | 2.3% | 2.3% | 2.3% |
| Butter | | | |
| Jojoba oil | 2% | 2% | 2% |
| Glyceryl caprylate | 0.5% | 0.5% | 0.5% |
| Lanol ™ 99⁽²⁾ | 3.5% | 3.5% | 3.5% |
| Cetearyl alcohol | 0.5% | 0.5% | 0.5% |
| Solubilizing agent | | | |
| 2-Octyl-1-dodecanol | 7% | 0% | 0% |
| Caprylic/capric | 0% | 7% | 0% |
| triglycerides | | | |
| Monopropylene glycol | 0% | 0% | 7% |
| Palmitoyl isoleucine⁽⁵⁾ | 1% | 1% | 1% |
| Thickening polymer | | | |
| Sepinov ™ EMT10⁽³⁾ | 0.7% | 0.7% | 0.7% |
| Aqueous phase | | | |
| Water | qs 100% | qs 100% | qs 100% |
| Sepicide ™ HB⁽⁴⁾ | 1% | 1% | 1% |

⁽¹⁾MONTANOV ™ L (INCI name C14-22 ALCOHOL/C12-20 ALKYL GLUCOSIDE), is a self-emulsifying composition sold by the company SEPPIC, consisting of a mixture of tetradecyl, hexadecyl, octadecyl, behenyl and arachidyl alcohols with dodecylpolyglucosides, tetradecylpolyglucosides, hexadecylpolyglucosides, octadecylpolyglucosides, arachidylpolyglucosides and behenylpolyglucosides.
⁽²⁾LANOL ™ 99 is the isononyl isononanoate sold by the company SEPPIC.
⁽³⁾SEPINOV ™ EMT 10 is a thickener comprising a crosslinked copolymer of sodium acryloyldimethyl taurate and of hydroxyethyl acrylate which is in the form of a powder and sold by the company SEPPIC.
⁽⁴⁾SEPICIDE ™ HB, which is a mixture of phenoxyethanol, methylparaben, ethylparaben, propylparaben and butylparaben, is a preservative sold by the company SEPPIC.
⁽⁵⁾The palmitoyl isoleucine is characterized by a melting point of 92° C.

The measurement of the melting point is carried out using a METTLER automatic apparatus equipped with a cell of FP81 type and linked to an FP90 control unit, sold by the company Mettler, which makes it possible to capture thermo-optical modifications of the substance studied: a sample of the substance to be analyzed is introduced into a glass capillary tube, which is placed in the measuring apparatus and heated with a defined heating rate. A photoelectric cell of the apparatus continually records the intensity of the transmitted light, that is to say of the transparency, of the sample. The melting point is then characterized by the temperature at which the transparency of the sample is detected by the photoelectric cell.

Half the amount of each emulsion thus prepared is then stored in an isolated climate-controlled chamber regulated at a temperature of 20° C. for one month. The other half of the amount of each emulsion thus prepared is stored in an isolated climate-controlled chamber regulated at a temperature of 45° C. for one month.

At the end of this one-month period, the appearance of each emulsion prepared, stored at 20° C. and at 45° C. is observed and the viscosity of the emulsions formed is also measured respectively at 20° C. and at 45° C. As regards the emulsions stored at 45° C. for one month, an amount of said emulsions is stabilized in an isolated climate-controlled chamber regulated at 20° C. for 24 hours and the viscosity is then measured.

The results obtained and observed are recorded in table 2 below.

TABLE 2
Characterization of emulsions E1 to E3

| | Emulsion E1 | | |
|---|---|---|---|
| | (according | | |
| | to the | Comparative | Comparative |
| | invention) | emulsion E2 | emulsion E3 |
| Appearance | Homogeneous: | Heterogeneous: | Heterogeneous: |
| after one month | absence of | presence of | presence of |
| at 20° C. | lumps and of | lumps and of | lumps and of |
| | crystals | crystals | crystals |
| Viscosity after | 110 000 mPa · s | 136 000 mPa · s | 120 000 mPa · s |
| one month at | | | |
| 20° C. | | | |
| (Brookfield | | | |
| RVT | | | |
| viscometer, | | | |
| spindle 7, | | | |
| speed 5 | | | |
| revolutions/ | | | |
| min) | | | |
| Viscosity after | 49 000 mPa · s | 47 000 mPa · s | 45 000 mPa · s |
| one month at | | | |
| 45° C. | | | |
| (Brookfield | | | |
| LVT | | | |
| viscometer, | | | |
| spindle 4, | | | |
| speed 6 | | | |
| revolutions/ | | | |
| min) | | | |
| Viscosity after | 95 000 mPa · s | 90 000 mPa · s | 90 000 mPa · s |
| one month at | | | |
| 45° C. | | | |
| and | | | |
| subsequent | | | |
| stabilization at | | | |
| 20° C. | | | |
| (Brookfield | | | |
| LVT | | | |
| viscometer, | | | |
| spindle 4, | | | |
| speed 6 | | | |
| revolutions/ | | | |
| min) | | | |

The results included in table 2 reveal that the oil-in-water emulsion (E1) comprising 2-octyl-1-dodecanol in the fatty phase is stable and homogeneous after storage for a period of one month at 20° C., whereas the emulsions (E2) and (E3) exhibit a heterogeneous appearance characterized by the presence of lumps, grains and crystals during the same storage period under the same conditions.

Furthermore, when the emulsion (E1) according to the invention was stored for one month at a temperature of 45° C. and then subsequently re-stabilized at 20° C., the difference in viscosity compared with storage at 20° C. for one month consists of a loss of 13.6% of the viscosity value at 20° C., whereas this loss increases to 33.8% for the emulsion (E2) and to 25% for the emulsion (E3), thus reflecting a better stabilization of the emulsion according to the invention.

Formulations

In the following formulae, the percentages are expressed as percentages by weight for 100% of the weight of the formulation.

### EXAMPLE 2

### “Volufirming” Firming Face Cream

Phase A:

| | |
|---|---|
| LANOL ™ P: | 1.5% |
| MONTANOV ™ 202: | 2.0% |
| MONTANOV ™ 82: | 1.0% |
| Squalane: | 7.0% |
| Polyisobutene | 10.0% |
| FLUIDANOV 20X | 1.0% |
| N-Palmitoyl isoleucine | 1.0% |

Phase B:

| | |
|---|---|
| Water | qs 100% |
| Chlorphenisin | 0.3% |
| Triethanolamine | qs pH 6.0 |
| Glycerol | 5.0% |

Phase C:

| | |
|---|---|
| Xanthan gum | 0.15% |

Phase D:

| | |
|---|---|
| SEPIPLUS ™ 400 | 0.8% |

Phase E:

| | |
|---|---|
| SEPICIDE ™ LD | 0.7% |
| Perfume/fragrance | 0.1% |

Preparation Process:

Introduce the ingredients of phase A separately into a reactor at a temperature of 85° C., and mix for 30 minutes at 85° C. with a mechanical mixer equipped with a paddle of “anchor” type.

Add phase D to phase A at a temperature of 85° C. with stirring and homogenize, still at 85° C.

Prepare phase B separately by adding the various ingredients to water. Bring phase B to 85° C. and add phase C to phase B with stirring at a temperature of 85° C.

Add phase (A+D) to phase (B+C) at a temperature of 85° C. and emulsify this mixture using a stirrer equipped with a rotor-stator at a speed of 4000 revolutions/minute for 4 minutes. Cool the mixture obtained while stirring with a spindle of “anchor” type at a speed of 100 revolutions/minute by cooling for 30 minutes so as to reach a temperature of 40° C. Then add phase E.

### EXAMPLE 3

### “Push-Up Potion” Firming Bust Cream

Phase A:

| Water | qs 100% |
|---|---|
| Glycerol | 3.0% |

Phase B:

| | |
|---|---|
| N-Palmitoyl isoleucine | 1.0% |
| Mineral oil | 6.0% |
| Butyrospermum Parkii (Shea Butter) | 1.5% |
| FLUIDANOV ™ 20X | 2.0% |
| Neopentyl glycol diethylhexanoate | 5.0% |
| MONTANOV ™ 202: | 2.0% |

Phase C:

| | |
|---|---|
| SIMULGEL ™ INS 100 | 1.0% |

Phase D:

| | |
|---|---|
| Phenoxyethanol and methylparaben and | 0.5% |
| propylparaben and methylisothiazolinone | |
| Perfume/fragrance | 0.3% |

Preparation Process:

Introduce the ingredients of phase B separately into a reactor at a temperature of 85° C., and mix for 30 minutes at 85° C. with a mechanical stirrer equipped with a paddle of “anchor” type.

Prepare phase A separately by adding the various ingredients to water. Bring phase A to 85° C. Add phase C to phase B with stirring at a temperature of 85° C.

Add phase (B+C) to phase A at a temperature of 85° C. and emulsify this mixture using a stirrer equipped with a rotor-stator at a speed of 4000 revolutions/minute for 4 minutes.

Cool the mixture obtained while stirring with a spindle of “anchor” type at a speed of 100 revolutions/minute by cooling for 30 minutes so as to reach a temperature of 40° C. Then add phase D.

### EXAMPLE 4

### Youthful Freshness Foundation Cream

Phase A:

| | |
|---|---|
| LANOL ™ P: | 1.5% |
| MONTANOV ™ 202: | 2.0% |
| MONTANOV ™ 82: | 1.0% |
| Caprylic/capric triglyceride: | 7.0% |
| Polyisobutene | 6.0% |
| 2-Octyl-1-dodecanol | 5.0% |
| Dimethicone | 2.0% |
| N-Palmitoyl glycine | 1.0% |

Phase B:

| | |
|---|---|
| Water | qs 100% |
| Triethanolamine | qs pH 5.0 |
| Glycerol | 5.0% |

Phase C:

| | |
|---|---|
| Xanthan gum | 0.15% |

Phase D:

| | |
|---|---|
| SEPIPLUS ™ 400 | 0.7% |

Phase E:

| | |
|---|---|
| Phenoxyethanol and ethylhexylglycerin | 1.0% |
| Perfume/fragrance | 0.1% |

Preparation Process:

Introduce the ingredients of phase A separately into a reactor at a temperature of 85° C., and mix for 30 minutes at 85° C. with a mechanical stirrer equipped with a blade of “anchor” type.

Add phase D to phase A at a temperature of 85° C. with stirring and homogenize still at 85° C.

Separately prepare phase B by adding the various ingredients to water. Bring phase B to 85° C. and add phase C to phase B with stirring at a temperature of 85° C.

Add phase (A+D) to phase (B+C) at a temperature of 85° C. and emulsify this mixture using a stirrer equipped with a rotor-stator at a speed of 4000 revolutions/minute for 4 minutes. Cool the mixture obtained while stirring with a spindle of “anchor” type at a speed of 100 revolutions/minute by cooling for 30 minutes so as to reach a temperature of 40° C. Then add phase E.

The definitions of the commercial products used in the examples are the following:

LANOL™ P (INCI name: Glycol Palmitate) is an ester used as an oily phase in the preparation of cosmetic compositions and is distributed by the company SEPPIC.

MONTANOV™ 202 (INCI name: Arachidyl Alcohol and Behenyl Alcohol and Arachidyl Glucoside) is a self-emulsifying composition sold by the company SEPPIC.

MONTANOV™ 82 (INCI name: Cetearyl Alcohol and Coco Glucoside) is a self-emulsifying composition sold by the company SEPPIC.

FLUIDANOV 20X (INCI name: Octyldodecanol and Octyldodecyl Xyloside) is a composition sold by the company SEPPIC.

SEPIPLUS™ 400 (INCI name: Polyacrylate-13 and Polyisobutene and Polysorbate 20) is a polymeric thickening composition which is in the form of a concentrated inverse emulsion (inverse latex), sold by the company SEPPIC.

SEPICIDE™ LD is a preservative containing phenoxyethanol, sold by the company SEPPIC.

SIMULGEL™ INS 100 (INCI name: Hydroxyethyl Acrylate/Sodium Acryloyldimethyl Taurate Copolymer and Isohexadecane and Polysorbate 60) is a polymeric thickening composition which is in the form of an inverse emulsion (inverse latex), sold by the company SEPPIC.

1. A composition in the form of an oil-in-water emulsion comprising, for 100% of its weight:
(1) from 95% to 50% by weight and more particularly from 90% to 70% by weight of a cosmetically acceptable aqueous phase (P1), and
(2) from 5% to 50% by weight and more particularly from 10% to 30% by weight of a fatty phase (P2), characterized in that said fatty phase (P2) comprises, for 100% of its weight:
a) from 0.2% to 60% by weight, more particularly from 2% to 20% by weight, of at least one N-acylated amino acid derivative of formula (I):
R₁—C(═O)—NH—CH—(R′₁)—C(═O)—OH (I)
in which R₁ represents a saturated or unsaturated, linear or branched, aliphatic hydrocarbon-based radical comprising from seven to seventeen carbon atoms, and R′₁ represents a hydrogen atom or a hydrocarbon-based radical chosen from methyl, benzyl, isopropyl, 1-methylpropyl or isobutyl radicals, and
b) from 0.2% to 80% by weight, more particularly from 2% to 40% by weight, of a solubilizing agent comprising, for 100% of its weight:
(i) from 5% to 100% by weight of at least one compound of formula (II):
R—OH (II)
in which R represents a branched alkyl radical CH(CₙH₂ₙ₊₁)(CₘH₂ₘ₊₁)—CH₂— in which m is an even integer between 6 and 18, n is an even integer between 4 and 18, and the sum n+m is greater than 10, and
(ii) from 0% to 95% by weight of at least one compound of formula (III):
R′O—(X)ₚ (III)
in which p represents a decimal number between 1 and 5, X represents the xylose residue and R′, which may be identical to or different than the radical R as defined above, represents a branched alkyl radical CH(CₙH₂ₙ₊₁)(CₘH₂ₘ₊₁)—CH₂— in which m is an even integer between 6 and 18, n is an even integer between 4 and 18, and the sum n+m is greater than 10;
c) from 5% to 99.4% by weight, more particularly from 5% to 94% by weight, of one or more oils and/or of one or more waxes,
d) from 0.2% to 80% by weight, more particularly from 2% to 40% by weight, of an emulsifying system (A) comprising, for 100% of its weight:
(i) from 10% to 60% by weight of at least one of the elements of the group made up of the alkylpolyglycosides of formula (IV):
R₂—O-(G)ₓ-H (IV)
in which the radical R₂ represents a saturated linear aliphatic radical comprising from twelve to twenty-two carbon atoms, G represents the residue of a reducing sugar chosen from the group consisting of glucose, xylose and arabinose, and x represents a decimal number greater than or equal to 1 and less than or equal to 5, and
(ii) from 40% to 90% by weight of at least one fatty alcohol of formula (V):
R₃—OH (V)
in which the radical R₃, which may be identical to or different than the radical R₂ as defined above, represents a saturated linear aliphatic radical comprising from 12 to 22 carbon atoms.

2. The composition as defined in claim 1, characterized in that, in formula (I), the radical R₁—C(═O)— represents a radical chosen from octanoyl, decanoyl, dodecanoyl, tetradecanoyl, hexadecanoyl, octadecanoyl or octadecenoyl radicals.

3. The composition as defined in claim 1, characterized in that, in formula (I), the radical R′₁ represents a hydrogen atom or a hydrocarbon-based radical chosen from methyl, isopropyl, 1 methylpropyl or isobutyl radicals.

4. The composition as defined in claim 3, characterized in that, in formula (I), the radical R′₁ represents a hydrogen atom or a hydrocarbon-based radical chosen from methyl, 1-methylpropyl or isobutyl radicals.

5. The composition as defined in claim 4, in which the N-acylated amino acid derivative of formula (I) is N-palmitoyl alanine, N-palmitoyl glycine, N-palmitoyl leucine, N-palmitoyl isoleucine or N-cocoyl alanine.

6. The composition as defined in claim 1, for which, in formulae (II) and (III), the radicals R and R′, which may be identical or different, represent a radical chosen from 2-hexyldecyl, 2-hexyldodecyl, 2-octyldecyl, 2-octyldodecyl and 2-decyltetradecyl radicals.

7. The composition as defined in claim 1, characterized in that it also comprises, for 100% of its weight, from 0.1% to 5% by weight, more particularly from 0.1% to 2% by weight, of a thickening and/or gelling polymer.

8. A process for preparing the composition as defined in claim 1, characterized in that it comprises:
at least one step a) of preparing said fatty phase (P2) by mixing, for 100% by weight of said phase (P2):
a) from 0.2% to 60% by weight, more particularly from 2% to 20% by weight, of at least one N-acylated amino acid derivative of formula (I), with
b) from 0.2% to 80% by weight, more particularly from 2% to 40% by weight, of said solubilizing agent;
c) from 5% to 99.4% of one or more oils and/or of one or more waxes and with
d) from 0.2% to 80% by weight, more particularly from 2% to 40% by weight, of said emulsifying system (A); and
e) optionally a thickening and/or gelling polymer, in the amount required to obtain, in said composition, from 0.1% to 5% by weight, more particularly from 0.1% to 2% by weight, of said thickening and/or gelling polymer,
at least one step b) of emulsifying from 5% to 50% by weight of said fatty phase P2 in from 95% to 50% by weight of said aqueous phase P1.

9. Method of cosmetically treating the skin or mucus membranes of a human body, which comprises topically applying to the skin or the mucus membranes of the human body an effective amount of the composition as defined in claim 1.

10. The composition as defined in claim 2, characterized in that, in formula (I), the radical R′₁ represents a hydrogen atom or a hydrocarbon-based radical chosen from methyl, isopropyl, 1-methylpropyl or isobutyl radicals.

11. The composition as defined in claim 10, characterized in that, in formula (I), the radical R′₁ represents a hydrogen atom or a hydrocarbon-based radical chosen from methyl, 1-methylpropyl or isobutyl radicals.

12. The composition as defined in claim 11, in which the N-acylated amino acid derivative of formula (I) is N-palmitoyl alanine, N-palmitoyl glycine, N-palmitoyl leucine, N-palmitoyl isoleucine or N-cocoyl alanine.

13. The composition as defined in claim 2, for which, in formulae (II) and (III), the radicals R and R′, which may be identical or different, represent a radical chosen from 2-hexyldecyl, 2-hexyldodecyl, 2-octyldecyl, 2-octyldodecyl and 2-decyltetradecyl radicals.

14. The composition as defined in claim 2, characterized in that it also comprises, for 100% of its weight, from 0.1% to 5% by weight, more particularly from 0.1% to 2% by weight, of a thickening and/or gelling polymer.
